(19) United States
(12) Patent Application Publication
(10) Pub. No.: US 2023/0089681 A1
(43) Pub. Date: Mar. 23, 2023

(54) USE OF A COMBINATION OF SULFICANT COMPOUNDS WITH A LIQUID CARRIER AS A SELF EMULSIFYING DRUG DELIVERY SYSTEM

(71) Applicant: Eric Marchewitz, ake Orion, MI (US)

(72) Inventor: Eric Marchewitz, ake Orion, MI (US)

(21) Appl. No.: 17/950,017

(22) Filed: Sep. 21, 2022

Related U.S. Application Data
(60) Provisional application No. 63/246,577, filed on Sep. 21, 2021.

(51) Int. Cl. A61K 47/10; A61K 47/46; A61K 47/26; A61K 47/40

(52) U.S. Cl. CPC A61K 47/10; A61K 47/46; A61K 47/26; A61K 47/40

(57) ABSTRACT

This invention relates a method of orally administering surfactant and solubilizing agent compounds to a human or animal for the purpose of emulsifying drugs/natural compounds to increase absorption into the body.

### FIELD OF THE INVENTION

This invention relates a method of orally administering surfactant and solubilizing agent compounds to a human or animal for the purpose of emulsifying drugs/natural compounds to increase absorption into the body.

### BACKGROUND OF THE INVENTION

Current methods for traditional oral administration for the delivery of drugs and dietary supplements into the body include non-soluble compounds which are not absorbed easily by the body and result in the loss of the associated benefits being sought. The result is that the amount of the compound that must be taken to achieve the desired benefit is far more than it should be . For example, the absorption of Berberine is 0.36%. This results in a dose of 300 mgs to get 1mg delivered into the body. Self-emulsifying drug delivery systems (SEDDS) are well established in the literature to provide a vastly superior mechanism for the delivery of drugs and dietary ingredients compared with traditional administration. However, there exist a need for specific combinations of carriers with a series of surfactants and solubilizing agents to solubilize the drug or natural ingredient in a more efficient manner.

Disclosed in this invention is a series of nethods for combining a carrier, for example, ethanol, to dissolve a drug or natural ingredient compound, with a series of surfactants and solubilizing agents in a solution of distilled water along with Glycerol for the purpose of emulsifying drugs/natural compounds to increase absorption in the intestines.

### SUMMARY OF THE INVENTION

In accordance with a first aspect, methods are disclosed for methods for combining a carrier, for example, ethanol, to dissolve a drug or natural ingredient compound, with a series of surfactants and solubilizing agents and dried or added in a solution of distilled water along with Glycerol for the purpose of emulsifying drugs/natural compounds to increase absorption in the stomach intestines of humans and animals.

From the foregoing disclosure and the detailed description set forth below of various preferred embodiments of the invention it will be apparent to those skilled in the art that the disclosed invention provides a significant advance in the methods of administering oral compounds, such as drug or dietary supplement compounds, with improved self-emulsifying drug delivery systems. Our unique formula is theorized to provide a 4000% increase over traditional oral administration of drugs and dietary supplements. Numerous natural compounds such, as Berberine, have very poor oral absorption. Berberine, which can lower blood sugar and extend lifespan has only a 0.306% absorption in capsule for taken with water. SEDDS can increase the blood levels of Berberine without taking a toxic dose. Additional features and advantages of various preferred embodiments will be better understood in view of the detailed description provided below.

### DETAILED DESCRIPTION

It will be apparent to those skilled in the art, that is, to those who have a knowledge or experience in this area of technology that many variations are possible for the method of orally administering self-emulsifying drug delivery systems into humans and/or animals. The methods and formulations disclosed herein are new and have a distinct advantage over existing oral delivery methods of drugs and dietary supplements.

All of the compound formulations disclosed herein would be administered orally or transdermally, with a specific carrier formulation combined with an active surfactant and a solubilizing agent. The carrier solution might be composed of one or more of an Ethanol, Isopropyl Alcohol, Water, and/or Oleic Acid solution. The surfactants might include one or more compound including fractionated coconut oil, Propylene Glycol, Polyethylene Glycol, Polysorbate 80, and/or Polysorbate 60. The solubilizing agent might include one or more compound including Beta Cyclodextrin, Hydroxypropyl-Cyclodextrin, Methyl-Cyclodextrin, and/or Dextrose. In the preferred embodiment these various compounds would preferably be administered orally mixed with a liquid carrier in appropriate unit doses.

The preferred amount of each of the three active ingredients within the formulation that is to be orally administered would depend on various factors such as the specific drug or dietary supplement that needs to be solubilized to obtain the desired result. The concentration of the active surfactant in the formulation might be between 20% and 1%. It might be 12% and 5%. The preferred concentration would be 7.5%. The concentration of the active solubilizing in the formulation might be between 20% and 1%. It might be between 10% and 1%. The preferred concentration would be 3%.

### Example formulation one:

A 1000 ml solution containing

70% Ethanol as a carrier—700 ml

15% Propylene Glycol—150 ml

7.5% Fractionated Coconut Oil—75 ml

7.5% Polysorbate 80—75 ml

20 g HydroxyPropyl Cyclodextrin

### Example formulation two:

A 10000 ml solution containing:

65% Ethanol—6500 ml

20% Polyethylene Glycol—2000 ml

10% Fractionated Coconut Oil—1000 ml

10% Polysorbate 60—1000 ml

1 kg—Beta Cyclodextrin

From the foregoing disclosure and detailed description of certain preferred embodiments, it will be apparent that various modifications, additions, and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method of administering orally or transdermally, a combined solution of surfactants and solubilizing agents for the purpose of emulsifying drugs/natural compounds to increase absorption in the stomach intestines of humans and animals.

2. The method of claim 1 wherein the surfactants consist of at least one of at least one or more of a Fractionated Coconut Oil, Propylene Glycol, Polyethylene Glycol, Polysorbate 80, and/or Polysorbate 60 Compound.

3. The method of claim 1 wherein the solubilizing agents consist of at least one or more of a Beta-Cyclodextrin, Hydroxypropyl-Cyclodextrin, Methyl-Cyclodextrin, and/or Dextrose.

4. The method of claim 1 wherein the carrier solution consists of at least one or more of an Ethanol, Isopropyl Alcohol, Water, and/or Oleic Acid.
